(19) United States
(12) Patent Application Publication
(10) Pub. No.: US 2008/0145887 A1
(43) Pub. Date: Jun. 19, 2008

(54) CYTOLOGICAL FILTER WITH DATA STORAGE

(75) Inventor: Robert MacIndoe, Upton, MA (US)

Correspondence Address: VISTA IP LAW GROUP LLP, 12930 Saratoga Avenue, Suite D-2, Saratoga, CA, 95070, US

(73) Assignee: CYTYC CORPORATION, Marlborough, MA (US)

(21) Appl. No.: 11/613,171

(22) Filed: Dec. 19, 2006

(51) Int. Cl. C12N 1/02; C12M 1/00

(52) U.S. Cl. 435/29; 435/283.1; 435/286.5

(57) ABSTRACT

A read/write storage device is attached to a cytological filter used to collect cells from a container having a solution carrying a cytological specimen in the solution. Data including a flow rate of a first fluid (e.g., air) through the filter is stored in the data storage device, and may be retrieved as needed to determine a corresponding baseline rate at which a vacuum level decays as a cell-free solution is sampled. Once a baseline flow rate of a solution through the filter is established, cells of the specimen can be collected by the filter. As cells are collected, filter coverage determinations can be made by comparing the measured rate of vacuum decay as cells are collected relative to the baseline rate of vacuum decay determined from the data read from the filter storage device.

### FIELD OF INVENTION

The present invention relates to the preparation of biological specimens and, more particularly, to storing data and accessing data to and from a cytological filter.

### BACKGROUND

Medical professionals and technicians often analyze biological specimen slides thereon in order to analyze whether a patient has or may have a particular medical condition or disease. For example, a cytological specimen slide may be prepared and examined for the presence of malignant or pre-malignant cells as part of a Papanicolaou (Pap) smear test, or other cancer detection tests.

Referring to FIGS. 1-4, one known automated slide preparation system includes a container or vial 10 that holds a cytological specimen 12. The specimen 12 includes tissue and cells 14 (generally, “cells” 14). The system also includes a filter 20, a valve 30 and a fixed volume vacuum chamber 40. Cells 14 are dispersed within a fluid, liquid, solution or transport medium 16, such as a preservative solution. One known preservative solution is PreserveCyt, available from Cytyc Corporation, 250 Campus Drive, Marlborough, Mass. 01752 (www.cytyc.com).

One end of the filter 20 is disposed in the liquid 16, and the other end of the filter 16 is coupled to a fixed volume vacuum chamber 40 through the valve 30. Opening the valve 30 applies vacuum 42 to the filter 20 which, in turn, draws liquid 16 up into the filter 20. Cells 14 in the drawn liquid 16 are collected by a face or bottom 22 of the filter 20, as shown in FIG. 2. Referring to FIGS. 3 and 4, collected cells 14 can be applied to a cytological specimen carrier 50, such as a slide, by bringing the filter 20 in contact with the slide 50.

In this system, the vacuum chamber 40 is a fixed volume vacuum chamber. Thus, the vacuum level in the chamber 40 decreases from an initial level to a lower level as cells 14 are collected by the filter 20. The rate at which the vacuum level decays from an initial level (e.g., 90% of maximum vacuum) to a lower level (e.g., 60% of maximum vacuum) is monitored by reading a vacuum level indicator 44 or other device over time. The rate of vacuum decay indicates the amount of cell coverage on the filter 20. The vacuum level decays faster when the filter 20 has no cells 14 or only a few cells 14 compared to when the filter 20 has collected a larger number of cells 14.

Determining whether the filter 20 has sufficient cell coverage is based on how fast the vacuum level decays from an initial or baseline decay rate (fastest rate of decay) to a lower, threshold decay rate. For this purpose, in known systems, each filter 20 must be tested prior to processing to determine the initial baseline rate of vacuum decay when vacuum 42 is applied to the filter 20 to draw solution 16 that is free or substantially free of cells 14.

FIG. 5 illustrates one manner in which a filter 20 is tested to determine a baseline rate of vacuum decay when sipping cell-free fluid 16. Vacuum 42 is applied to the filter 20 to sip fluid 16 from the vial 10. As fluid 16 is drawn up through the filter 20, cells 14 are collected by the filter 20. The valve 30 or another connection can then be adjusted so that positive pressure 62 from pressure source 60 is applied to the filter 20. As a result, cells 14 that were collected by the filter 20 during vacuum 42 are blown or pushed from the filter 20 by positive pressure 62. Liquid 16 that was previously drawn up into the filter 20 is also pushed out from the bottom of the filter 20. Following application of positive pressure 62, liquid 16 near the bottom of the filter 20 is free or substantially free of cells 14. The positive pressure 62 is deactivated or disconnected, and vacuum 42 is applied again to the filter 20 to sip cell-free solution 16 below the filter 20. The rate at which the vacuum level decays during these sips of cell-free solution 16 is the baseline rate of vacuum decay for that particular filter 20. This process is performed for each filter 20 to be used.

Referring to FIG. 6, having established the baseline vacuum decay rate, the degree of cell coverage on a filter 20 is determined by comparing the rate at which vacuum decays during collection of cells 14 relative to the determined baseline rate of vacuum decay. For example, sufficient cell coverage is determined to be obtained when the measured rate of vacuum decay drops below a threshold, e.g., a 20% reduction relative to the baseline vacuum decay rate, or 80% of the baseline rate.

While known systems and methods have been used effectively in the past, they can be improved. It would be advantageous to have baseline vacuum decay rates and other filter data that is readily available without having to perform additional and time consuming sipping tests for each filter as shown in FIG. 5. It would also be desirable to be able to obtain data that can be used to determine baseline vacuum decay rates from directly from the filter itself.

### SUMMARY

In accordance with one embodiment of the invention, an apparatus for preparing a cytological specimen includes a cytological filter with an attached read/write data storage device, wherein a flow rate of a first fluid through the cytological filter may be stored in and retrieved from the read/write data storage device. The filter may further comprise an encoder and decoder, wherein the encoder may be used for storing the flow rate of the first fluid through the cytological filter to the read/write data storage device, and the decoder may be used for reading the flow rate of the first fluid through the cytological filter from the read/write data storage device.

An alternative embodiment is directed to a method for processing a cytological specimen. The method includes providing a cytological filter and storing a flow rate of a first fluid through the cytological filter to a read/write storage device attached to or embedded in the cytological filter. The method also includes reading the flow rate of the first fluid through the cytological filter from the read/write storage device and determining an amount of cytological filter coverage by collected cells of the cytological specimen using the flow rate data read from the read/write storage device.

Another embodiment is directed to a method for processing a cytological specimen and includes providing a cytological filter and storing a flow rate of a first fluid through the cytological filter to a read/write storage device attached to or embedded in the cytological filter. The method further includes reading the flow rate of the first fluid through the cytological filter from the read/write storage device and converting the flow rate of the first fluid through the cytological filter to a rate of decay of a vacuum during flow of a second fluid through the cytological filter. The second fluid hold the cytological specimen. The rate of decay of the vacuum is then used to determine an amount of cytological filter coverage.

In various embodiments, the cytological filter to which a read/write data storage device is attached includes a body and a membrane. The read/write data storage device can be attached to or embedded in the body, which supports a membrane that is used for collecting cells of the cytological specimen.

In various embodiments, the first fluid is air, and the specimen can be stored in a second fluid, such as a preservative solution, that is different than the first fluid.

Further, in various embodiments, the read/write data storage device can be a bar code, a data matrix, a radio frequency identification device, a magnetic storage device, an optical storage device or other suitable read/write device. The particular encoder and decoder that are used may depend, for example, on the type of data stored and retrieved and the type of read/write data storage device.

In addition to the flow rate of the first fluid, other data can also be stored to, and retrieved from, the read/write data storage device attached to the filter. For example, a type of cytological filter can also be stored in, and retrieved from, the read/write data storage device. A filter type can be, for example, gynecological, non-gynecological or urinary. Additionally, an expiration date of the cytological filter can be stored in, and retrieved from, the read/write data storage device.

In various embodiments, the flow rate data retrieved from the read/write data storage device can be converted into another parameter, such as a rate of decay of a vacuum using, for example, a look-up table or graph. Further, the other parameter can involve a different fluid. Thus, while the flow rate data involves a first fluid, such as air, the rate of decay of vacuum can be the rate of decay of vacuum involving a second fluid, such as a fluid that holds the cytological specimen. This vacuum decay rate can be used as a baseline rate of decay of a vacuum as a cell-free portion of the second fluid flows through the cytological filter. As cells are collected by the filter, the vacuum decay rate changes, and the amount of filter coverage can be determined by comparing the actual decay rate to the baseline decay rate that was determined using the flow rate data store to, and retrieved from, the read/write data storage device.

Other aspects of embodiments are described herein and will become apparent upon reading the following detailed description with reference to the accompanying drawings.

### BRIEF DESCRIPTION OF THE DRAWINGS

Referring now to the drawings in which like reference numbers represent corresponding parts throughout and in which:

FIG. 1 illustrates a known system and method using a cytological filter for collecting cells to be applied to a specimen slide;

FIG. 2 is a bottom view of a face of a known cytological filter with collected cells;

FIG. 3 illustrates a known method of applying cells collected by a cytological filter to a specimen slide;

FIG. 4 shows a specimen slide having cells applied by a cytological filter;

FIG. 5 illustrates a known method of obtaining a baseline rate at which a vacuum level decays over time;

FIG. 6 is a chart illustrating determining the amount of cytological filter coverage based on measured vacuum decay rates relative to a baseline decay rate as determined by the method shown in FIG. 5;

FIG. 7 is a flow chart illustrating a method of storing data to a cytological filter according to one embodiment;

FIG. 8 illustrates a read/write device attached to an outer surface of a cytological filter according to one embodiment;

FIG. 9 illustrates a read/write device embedded within a wall of a cytological filter according to one embodiment;

FIG. 10 illustrates a bar-code attached to an outer surface of a cytological filter according to one embodiment;

FIG. 11 a two-dimensional bar-code or matrix attached to an outer surface of a cytological filter according to one embodiment;

FIG. 12 is a diagram of a system for storing data to, and retrieving data from, a filter having a read/write storage device attached thereto, an encoder and a decoder according to one embodiment;

FIG. 13 is a diagram of a system that includes a converter for translating decoded data into another format or parameter according to one embodiment;

FIG. 14 further illustrates use of a converter according to one embodiment;

FIG. 15 illustrates a look-up table for converting data read from a read/write device attached to a cytological filter to data applicable for collecting cells in a solution according to one embodiment;

FIG. 16 generally illustrates a graph for determining baseline rates of vacuum decay corresponding to a flow rate data value;

FIG. 17 illustrates an example of a graph that can be used with various embodiments; and

FIG. 18 is a flow chart of a method of using a filter having a read/write device to prepare a cytological specimen according to one embodiment.

### DETAILED DESCRIPTION OF THE ILLUSTRATED EMBODIMENTS

Embodiments of the invention improve upon known slide preparation systems by providing an apparatus, system and method that can advantageously provide data about a cytological filter directly from the filter. This filter data can be used to determine baseline rates of vacuum decay. A determination of when sufficient cells and/or tissue have been collected by a filter can be performed by comparing rates of vacuum decay while cells are being collected relative to the determined baseline rate of decay.

For example, a read/write storage device can be attached to the filter. Data relating to flow rates of fluids through the filter can be stored to, and retrieved from, the storage device. This flow rate data can be used to determine a different parameter related to specimen slide preparation. According to one embodiment, the parameter is a baseline rate of vacuum decay corresponding to the rate at which a vacuum level decays when sipping cell-free preservative solution. When cells are collected by the filter from the preservative solution, the rate at which vacuum decays during cell collection relative to the baseline rate of vacuum decay indicates the degree of filter coverage by collected cells.

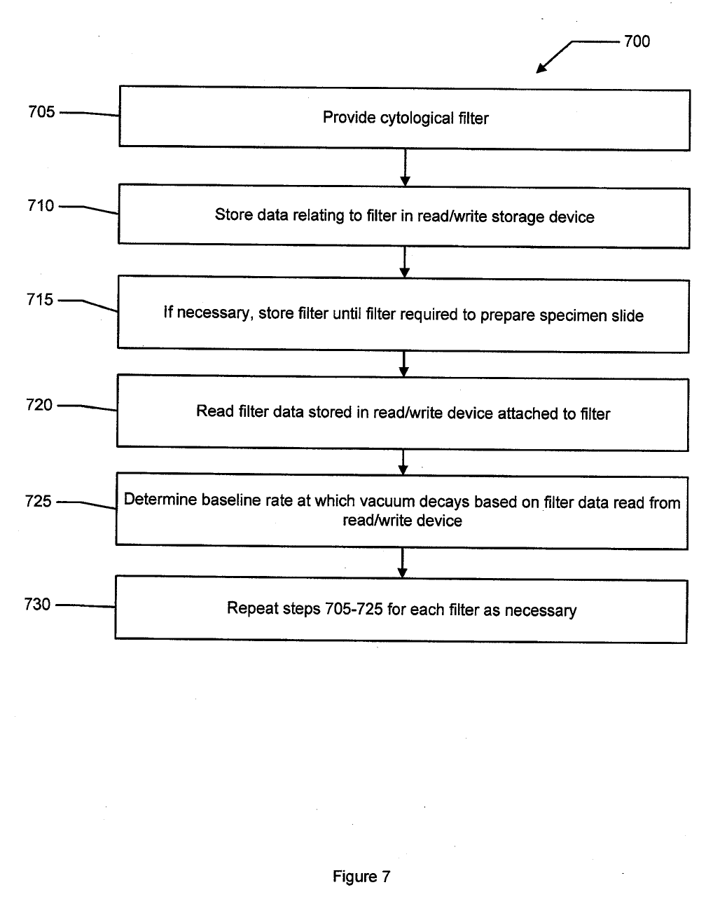

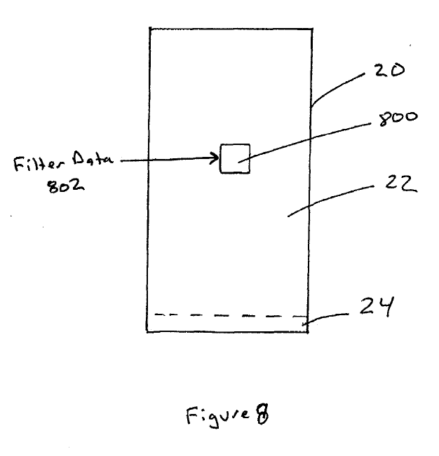

Further aspects of embodiments are described with reference to FIGS. 7-18. In the following description, reference is made to the accompanying drawings, which show by way of illustration specific embodiments in which the invention may be practiced. It is to be understood that other embodiments may be utilized as various changes may be made without departing from the scope of the invention.

Referring to FIG. 7, according to one embodiment, a method 700 of storing and retrieving cytological filter data includes providing a cytological filter that is used to collect cells from a biological or cytological specimen in step 705. This specification refers to cytological specimens for purposes of explanation, not limitation. In step 710, data or information relating to the filter is stored to a read/write storage device. The read/write storage device can be attached to or be a part of the filter. In step 715, if necessary, the filter can be stored until it is required, e.g., when the filter is to be used to prepare a specimen slide. When the filter is selected, in step 720, the previously stored filter data is read from the read/write device attached to the filter. In step 725, the retrieved data is used to determine a baseline rate at which a vacuum level decays over time as cell-free solution passes through the filter. In step 730, this process, steps 705-725, can be repeated for each filter that is to be used. Thus, embodiments advantageously read filter data from the filter itself, and use this information to determine another specimen preparation parameter, e.g., a baseline rate of vacuum decay, so that it is not necessary to perform preliminary “sipping” procedures (FIG. 5) for each filter.

According to one embodiment, data that is stored in the read/write storage device is the rate at which a first fluid flows through the filter. “Fluid” as used in this specification refers to both gaseous and liquid fluids. In one embodiment, the first fluid is air, e.g., at atmospheric pressure. This specification refers to the flow rate of air through the filter being stored in a read/write device attached to the filter, but the flow rate of other fluids through the filter can also be stored in the data storage device as necessary. The air flow rate data can be provided by filter manufacturers that make and test filters.

The stored air flow rate data can be retrieved from the read/write device to determine a corresponding baseline rate at which a different parameter varies. In one embodiment, the stored air flow rate is correlated to a baseline rate at which a vacuum level decays over time as a second fluid, which is different than the first fluid, flows through the filter. In one embodiment, the second fluid is a preservative solution in which the cytological specimen is stored. One exemplary preservative solution is PreservCyt, available from Cytyc Corporation. Persons skilled in the art will appreciate that other preservative solutions can be used, and that the second fluid can be a fluid or solution other than a preservative solution as necessary. In one embodiment, the first fluid is air and the second fluid is a preservative solution, and air flow rate data stored to the read/write storage device is used to determine a corresponding rate at which a vacuum level decays.

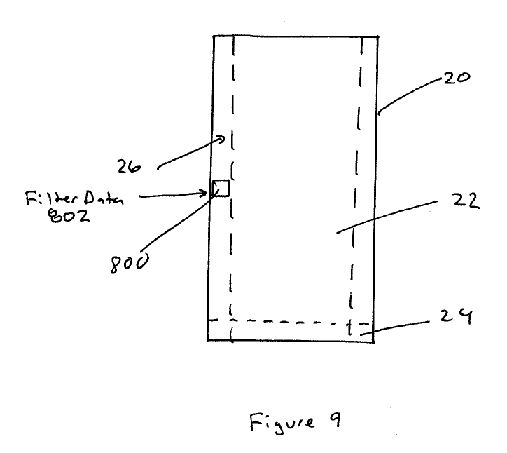

Referring to FIG. 8, a filter 20 includes a body 22 and a membrane or filter element 24. The filter 20 can be cylindrically shaped, and the body 24 can be plastic or other suitable material. One exemplary filter 20 is available from Cytyc Corporation. According to one embodiment, as shown in FIG. 8, the read/write storage device 800 (generally “storage device 800) for storing filter data 802 is attached to an outer surface of the filter 20, e.g., to the body 22 of the filter 20. The storage device 800 can be adhered or attached to the filter 20 using, for example, an adhesive, glue, tape, or a fastener. In the illustrated embodiment, the storage device 800 is attached to a side or body 22 of the filter 20.

Referring to FIG. 9, according to an alternative embodiment, the storage device 800 can be embedded or incorporated into the filter 20. In the illustrated embodiment, the storage device 800 is embedded or incorporated into a side wall 26 of the filter 20. The storage device 800 can be attached to or embedded in various parts of a filter 20. Thus, FIGS. 8 and 9 are provided for purposes of illustration, not limitation.

FIGS. 8 and 9 illustrate one storage device 800 attached to a filter 20. In alternative embodiments, different numbers of storage devices 800 can be attached to a filter 20 to provide enhanced data storage capabilities. Additionally, if desired, different types of storage devices 800 can be attached to the filter 20. Thus, references to a single storage device 800 are provided for purposes of explanation and illustration, not limitation.

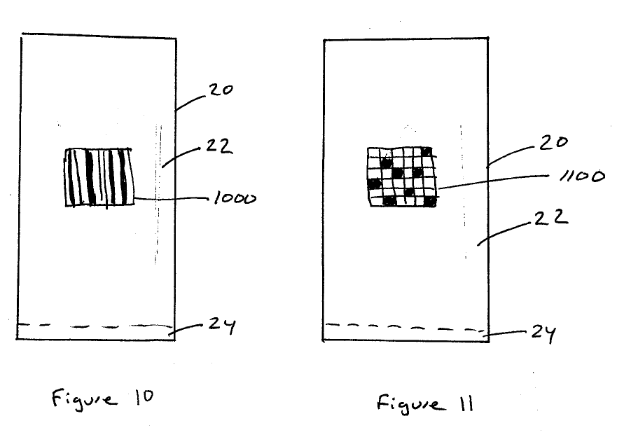

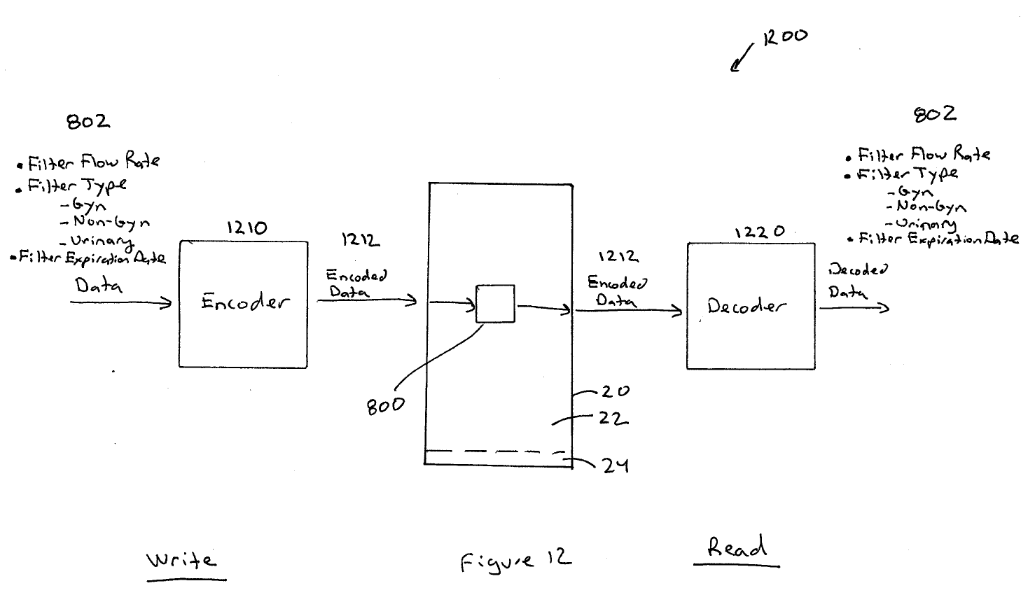

Referring to FIG. 10, according to one embodiment, the storage device 800 is a bar-code 1000 that is attached to the filter 20. In the illustrated embodiment, the bar-code 1000 is a one-dimensional bar-code. Other barcodes 1000 and symbologies can also be utilized. For example, in an further embodiment, the storage device 800 is a two-dimensional barcode 1100 or data matrix that is applied to the filter 20. In other embodiments, the storage device 800 can be a magnetic storage device, an optical storage device, a Radio Frequency Identification Device (RFID) and other suitable storage devices. Thus, FIGS. 10 and 11 are provided for purposes of illustration, not limitation, since various types and numbers of storage devices 800 can be used with embodiments depending on, for example, filter dimensions, design considerations, storage needs and read/write capabilities.

Referring to FIG. 12, according to one embodiment, a system 1200 for storing data 802 to and retrieving data 802 from a filter 20 includes a filter 20 having a read/write storage device 800 attached thereto, an encoder/writer (generally “encoder” 1210) and a decoder/reader (generally “decoder” 1220). Data 802 relating to the filter 20 is encoded 1212 or formatted and stored in the storage device 800. Data 802 is to be stored to the storage device 800 and can be flow rate data, e.g., the rate at which a first fluid, such as air, flows through the filter 20. The flow rate data 802 can be stored to the device 800 at various times including at the time the filter is manufactured or at a later time such as when the filter is tested. The data 802 can be provided by and stored in a storage device 800 by a filter manufacturer, testing facility, or other party.

Although this specification primarily refers to air flow rate data 802, other types of data 802 can also be stored to the storage device 800. For example, in one embodiment, as shown in FIG. 12, the data 802 can include a filter type (e.g., gynecological, non-gynecological or urinary) and a filter expiration date. For purposes of explanation, not limitation, this specification refers to flow rate data 802.

Once the flow rate data 802 is stored in the storage device 800 of the filter 20, the filter 20 can used immediately or be packaged and shipped for use at a later time. When the filter 20 is selected for use, the decoder 1220 reads encoded data 1212 from the storage device 800 and provides the flow rate data 802 to a cytotechnologist or to another system component as necessary. For example, if the flow rate data 802 was encoded as a barcode 1000, then a suitable detector/reader 1220 is a bar code reader. Similarly, if the flow rate data 802 was encoded and written to an optical storage device 800, then a suitable optical media detector/reader 1220 can read optical media. The encoder 1210 and decoder 1220 that are needed depend on the type of storage device 800 that is utilized.

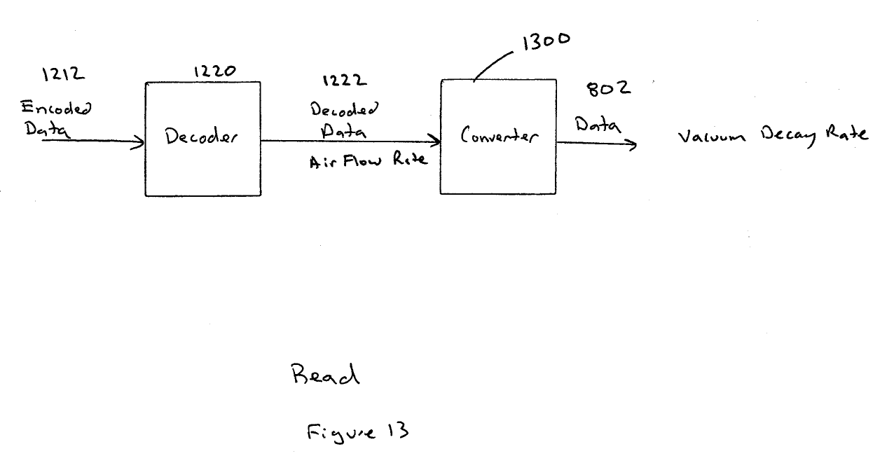

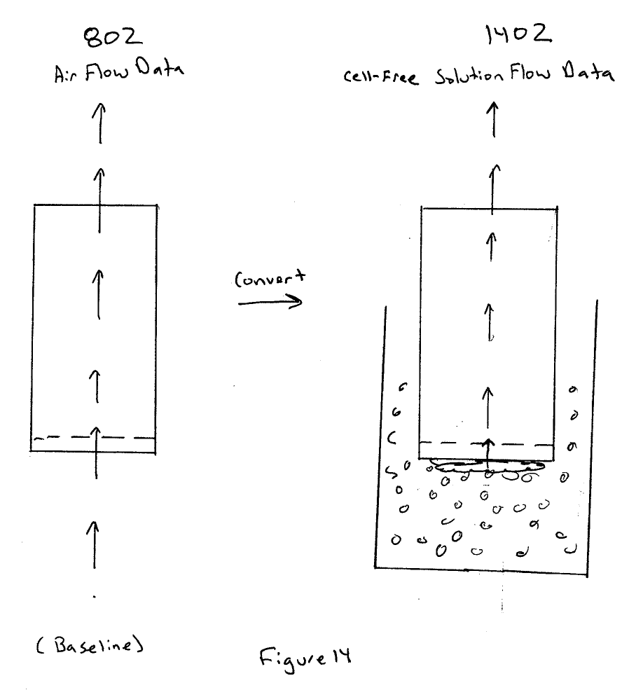

According to one embodiment, data read from the storage device 800 relates to a flow rate of a first medium or fluid, such as air, through the filter, and is used to determine another processing parameter, such as a vacuum decay rate. Thus, the data stored to and retrieved from the storage device relates to air flow rates, whereas the data used during collection of cells relates to a different parameter. For this purpose, referring to FIG. 13, the system 1200 can include a converter 1300 that receives the “air flow rate” data from the decoder 1220 and converts or translates decoded data 1222 into another format or parameter. A “converter” or “translator” as used in this specification is a device that converts, translates or relates a first type of data of a first fluid to a second type of data of a second fluid.

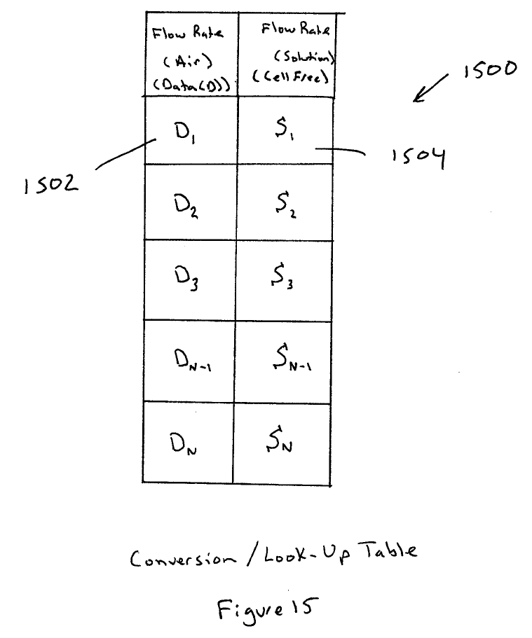

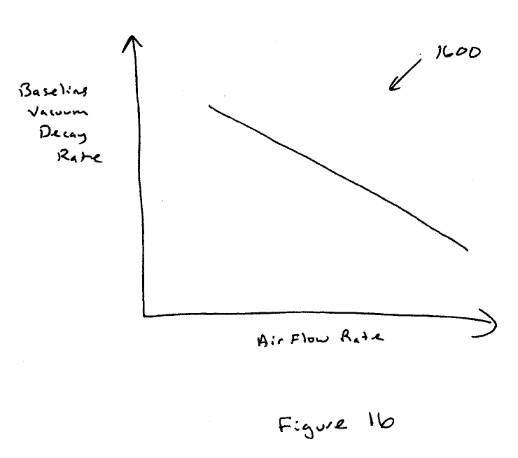

For example, in one embodiment, referring to FIG. 14, data 1222 output by the decoder/reader 1220 is the flow rate of a first liquid, such as air, through the filter 20. In order to use this data to establish a baseline rate of vacuum decay using that particular filter, 20 the liquid data is converted or translated into baseline vacuum decay rate data for a second liquid, which is different than the first liquid. For this purpose, referring to FIG. 15, the converter or translator can be a look-up table 1500. The table 1500 includes a series of flow rate data values 1502 and corresponding baseline rates of vacuum decay 1504.

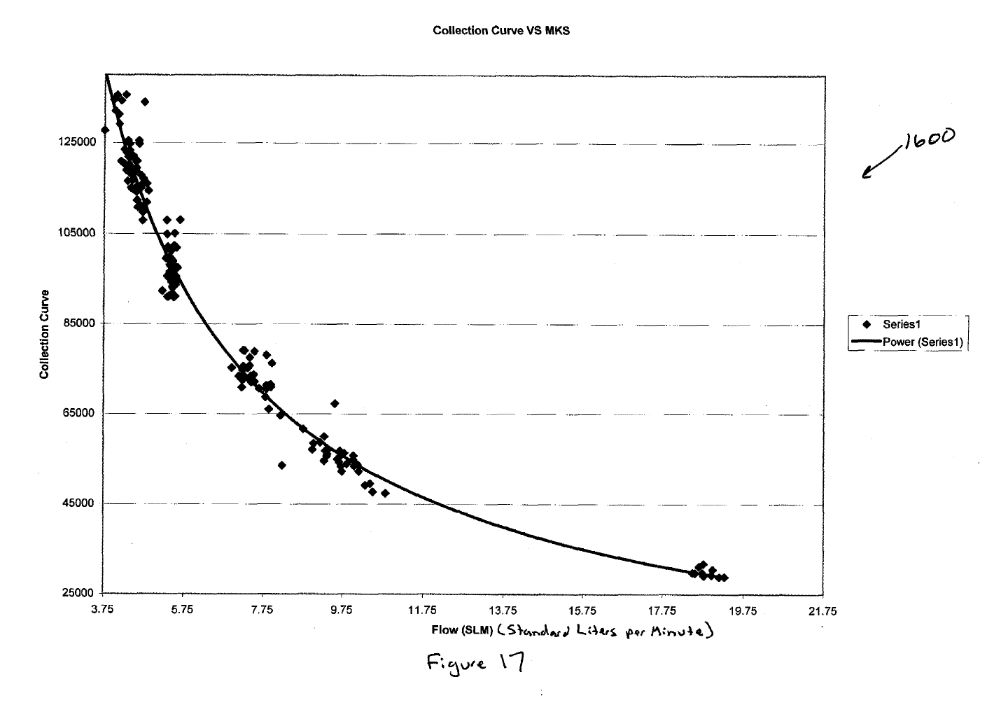

Alternatively, referring to FIG. 16, a graph or function 1600 can be used to determine baseline rates of vacuum decay corresponding to a particular flow rate data value. FIG. 17 illustrates an exemplary graph 1600. In FIG. 17, the “x” axis represents the flow rate of air through the filter as read from the read/write data storage device on the filter in terms of Standard Liters per Minute (SLM), and the “y” axis (Collection Curve) represents a corresponding decay time, in microseconds, from 90% of maximum vacuum to 60% of maximum vacuum inside the filter during a sip. “MKS” stands for MKS Instruments, Inc., the manufacturer of the flow meter that was used during this test. “Series 1” is the data acquired during the test, and “Power (Series 1) is the best-fit curve for the Series 1 data.

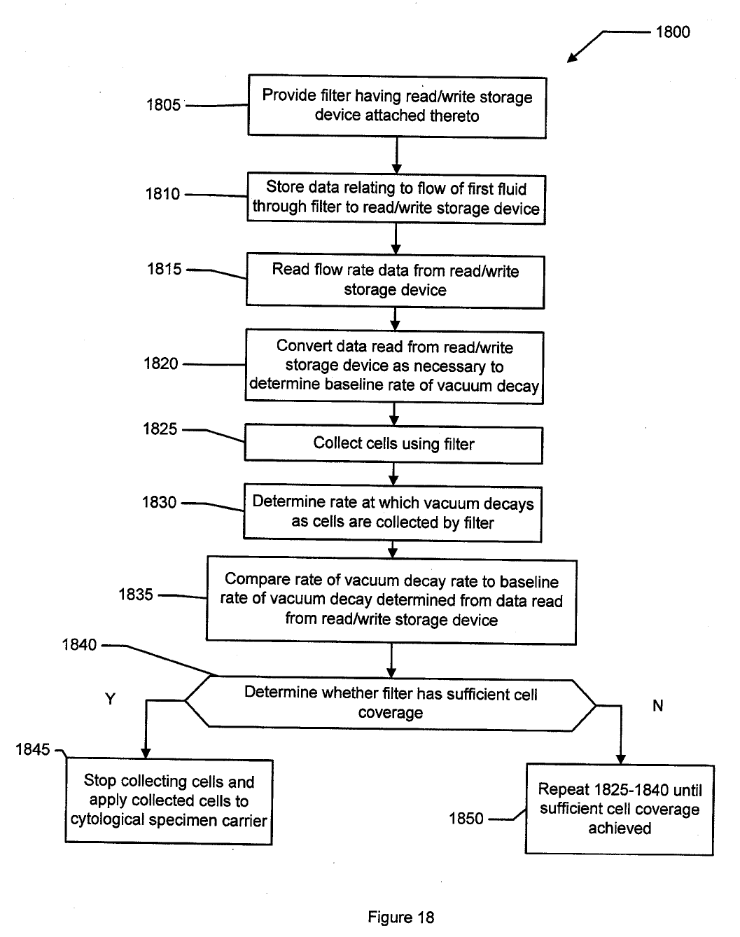

Referring to FIG. 18, a method 1800 of using a filter having a read/write device to prepare a cytological specimen includes providing a filter having storage device attached thereto (or attaching a storage device if necessary) in step 1805. In step 1810, data relating to a flow of a first fluid through the filter is stored to the storage device. If necessary, the filter can be stored until the filter is to be used to prepare a specimen, and when the filter is selected, in step 1815, the flow rate of the first fluid is read from the storage device. In step 1820, the data read from the read/write device is converted or translated as necessary, e.g., by converting flow rate or air through the filter to a baseline rate at which vacuum decays when a cell-free solution, such as cell-free PreservCyt solution, is sampled. In step 1825, cells of the cytological specimen are collected using the filter.

In step 1830, the rate at which vacuum decays as cells are collected is determined. In step 1835, the vacuum decay rate is compared to the baseline rate of vacuum decay that determined from the air flow rate data read from the storage device. In step 1840, based on this comparison, a determination is made whether the filter has sufficient cell coverage. If so, then in step 1845, collected cells can be applied to a cytological specimen carrier, such as a slide. If not, then in step 1850, additional cells can be collected by repeating steps 1825-1840 until sufficient cell coverage is obtained and cells can be applied to a cytological specimen carrier in step 1845.

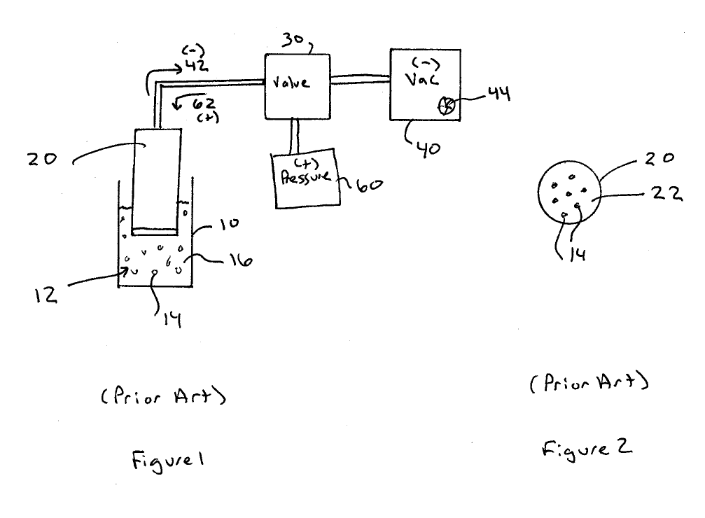

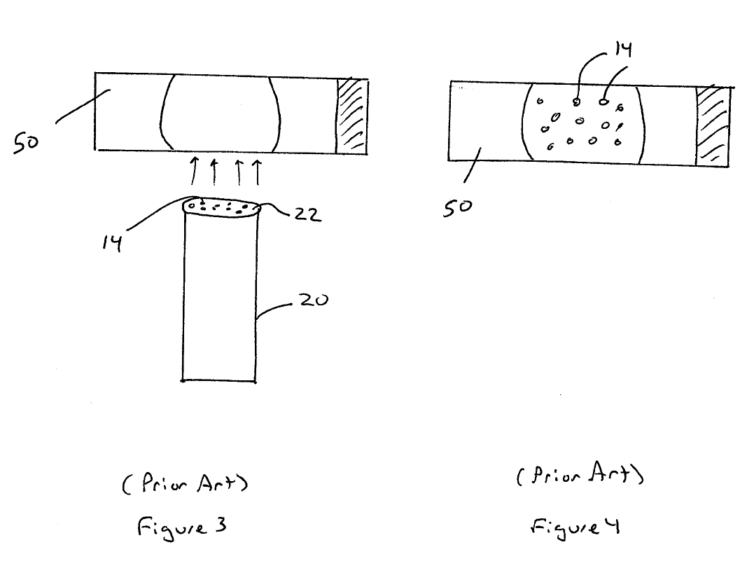

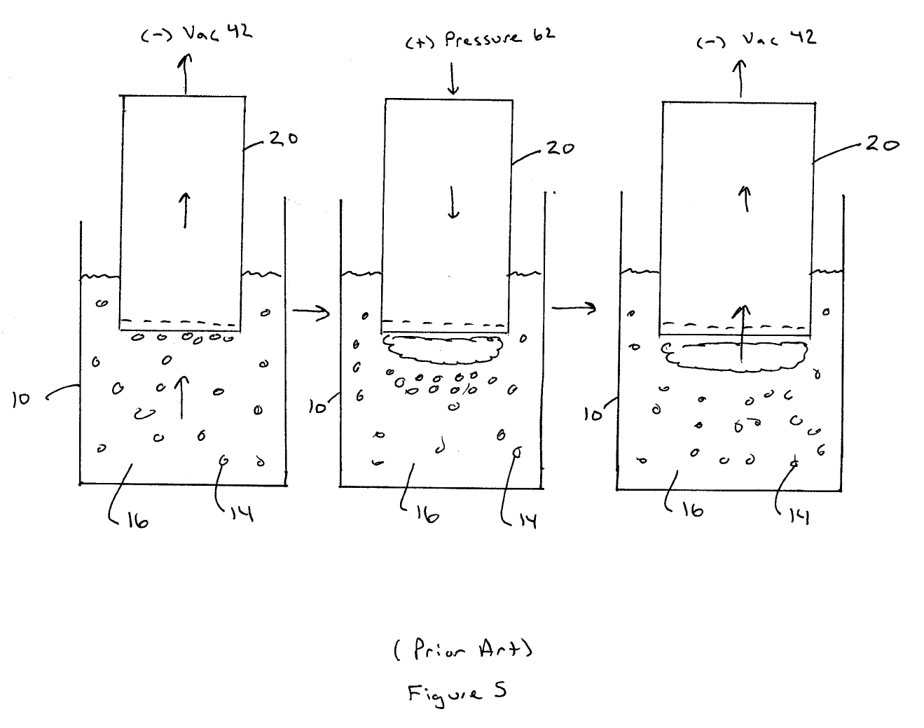

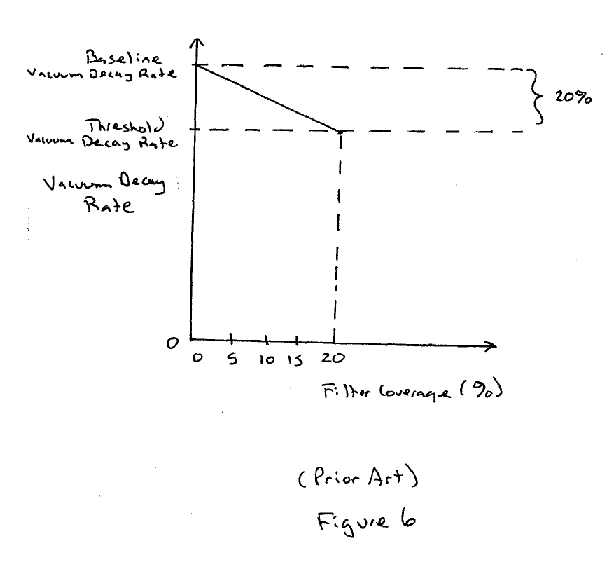

Thus, embodiments advantageously eliminate the need to perform separate sipping testing or other baseline testing of each filter in order to determine a baseline rate of vacuum decay. Instead, embodiments determine this baseline information directly from each filter and provide a more direct method of determining baseline decay rate information and provide more efficient slide preparation techniques. Further, embodiments achieve these advantages even thought each filter may vary since the filter variations are reflected in the flow rate of air through the filter data that is stored to, and retrieved from, the read/write storage device. Although particular embodiments have been shown and described, it should be understood that the above discussion is not intended to limit the scope of these embodiments. Further, embodiments have been described with reference to one known automated slide preparation system generally shown in FIGS. 1-5 and that includes a valve 30 and a fixed volume vacuum chamber 40. However, persons skilled in the art will appreciate that embodiments can also be used with other cytological specimen preparation systems.

For example, embodiments can be used with other automated slide preparation systems that use an open vacuum source and an air flow sensor, e.g., a system that includes a mass air flow sensor and a regulated open vacuum source. The regulated vacuum source provides a constant level of vacuum or negative pressure (rather than a decaying vacuum level as with a fixed volume vacuum source. A filter that is used in this system can include a read/write storage device attached thereto for storing data relating to flow rates of fluids, e.g., air, through the filter. The stored data is used to determine a baseline rate of vacuum decay corresponding to the rate at which a vacuum level decays when sipping cell-free preservative solution.

In this exemplary alternative system, the filter having the read/write data storage device is placed in a liquid containing a cytological specimen, the valve is opened, and a vacuum is applied to the filter. Cells are collected by the filter, and the air flow rate through the air flow sensor is measured while cells are collected by the filter. Having the air flow rate measurement, a determination is made whether the air flow rate has dropped to a certain level or has dropped by a certain amount relative to a flow rate that is determined from the information stored in the read/write data storage device attached to the filter. The data stored in the filter can be retrieved at the beginning of the process or when the data is actually needed. If the flow rate has dropped to a certain level, then the valve can be closed and the filter can be removed for further processing. If not, then the valve remains open so that the filter collects additional cells until the air flow rate measured by the air flow sensor has dropped to certain level or has dropped by a certain amount to indicate that the filter has sufficient cell coverage. Accordingly, persons skilled in the art will appreciate that embodiments can be used with various automated slide processing systems, including the system shown in FIGS. 1-5 and a system that includes a mass air flow sensor and a regulated open vacuum source.

Further, various changes and modifications may be made without departing from the spirit and scope of embodiments. Thus, embodiments are intended to cover alternatives, modifications, and equivalents that may fall within the spirit and scope of the claims.

What is claimed is:

1. An apparatus for preparing a cytological specimen, comprising:
a cytological filter; and
a read/write data storage, wherein a flow rate of a first fluid passing through the filter is stored in, and can be retrieved from, the read/write data storage device.

2. The apparatus of claim 1, the filter comprising a body, wherein the read/write data storage device is attached to, or embedded in, the filter body, the filter further comprising a membrane supported by the body, wherein cells of the cytological specimen are collected by the membrane.

3. The apparatus of claim 1, wherein the first fluid is air.

4. The apparatus of claim 1, wherein the cytological specimen is carried in a second fluid different than the first fluid.

5. The apparatus of claim 4, wherein the first fluid is air, and the second fluid is a preservative solution.

6. The apparatus of claim 1, wherein the read/write data storage device is selected from the group comprising a bar code, a data matrix, a radio frequency identification device, a magnetic storage device, and an optical storage device.

7. The apparatus of claim 1, wherein the filter comprises a filter type, and wherein the filter type is stored in, and can be retrieved from, the read/write data storage device.

8. The apparatus of claim 1, wherein the filter comprises an expiration date, and wherein the expiration date is stored in, and can be retrieved from, the read/write data storage device.

9. A system for preparing a cytological specimen, comprising:
a cytological filter;
a read/write data storage device;
an encoder for storing a flow rate of a first fluid through the filter in the read/write data storage device; and
a decoder for retrieving the flow rate of the first fluid through the filter from the read/write data storage device.

10. The apparatus of claim 9, the filter comprising a body, wherein the read/write data storage device is attached to, or embedded in, the filter body, the filter further comprising a membrane supported by the body, wherein cells of the cytological specimen are collected by the membrane.

11. The system of claim 9, further comprising a converter for converting the flow rate of the first fluid through the filter to a different parameter.

12. The system of claim 11, wherein the flow rate is converted into a rate of decay of a vacuum.

13. The system of claim 12, wherein the rate of decay of the vacuum corresponds to a rate of decay of a vacuum as a second fluid containing cytological specimen flows through the filter.

14. The system of claim 13, wherein the rate of decay of the vacuum corresponds to a baseline rate of decay of a vacuum as a cell-free portion of the second fluid flows through the filter.

15. The system of claim 11, wherein the converter comprises a look-up table or a graph.

16. A method for processing a cytological specimen, comprising:
storing a flow rate of a first fluid through a cytological filter to a read/write storage device attached to, or embedded in, the filter;
reading the flow rate from the read/write storage device; and
determining an amount of cellular material covering the filter based on the flow rate read from the read/write storage device.

17. The method of claim 16, wherein the first fluid is air.

18. The method of claim 16, wherein the amount of filter coverage is determined based at least in part on converting the flow rate of the first fluid through the filter to a corresponding rate of decay of a vacuum as a second fluid flows through the filter, the second fluid containing the cytological specimen, the first fluid being different than the second fluid.

19. The method of claim 18, wherein the first fluid is air, and the second fluid is a preservative solution.

20. The method of claim 18, wherein the rate of decay of the vacuum corresponds to a baseline rate of decay of a vacuum as a cell-free portion of the second fluid flows through the filter.
